(19) United States
(12) Patent Application Publication
(10) Pub. No.: US 2022/0323324 A1
(43) Pub. Date: Oct. 13, 2022

(54) COSMETIC COMPOSITION WITH ENHANCED COLOR STABILITY

(71) Applicant: Conopco, Inc., d/b/a UNILEVER, Englewood Cliffs, NJ (US)

(72) Inventors: Hasiba BEKTO, Bristol, CT (US); Lei HUANG, Trumbull, CT (US); Teanoosh MOADDEL, Watertown, CT (US)

(73) Assignee: Conopco, Inc., d/b/a UNILEVER, Englewood Cliffs, NJ (US)

(21) Appl. No.: 17/642,732

(22) PCT Filed: Oct. 14, 2020
(86) PCT No.: PCT/EP2020/078953

§ 371 Date: Mar. 14, 2022

(30) Foreign Application Priority Data
Oct. 25, 2019 (EP) 19205384.1

(51) Int. Cl. A61K 8/34; A61K 8/67; A61K 8/37; A61K 8/365; A61Q 19/08

(52) U.S. Cl. CPC A61K 8/347; A61K 8/671; A61K 8/37; A61K 8/365; A61K 8/375; A61K 8/675; A61Q 19/08

(57) ABSTRACT

The present invention is directed to a personal care composition containing resorcinols. An effective way to improve the color stability of compositions comprising resorcinols is desired. It has been found that alkylresorcinols and retinoids in combination with a compatible oil and specific ratios of antioxidants provide for improved color stability of the resorcinols. The invention thus relates to a composition comprising a synergistic combination of butylated hydroxytoluene and dilauryl thiodipropionate with alkylresorcinols and retinoids.

### FIELD OF THE INVENTION

The present invention is in the field of personal care compositions; in particular skin-benefit compositions. More particularly, the present invention relates to a cosmetic composition containing oil-soluble alkylresorcinols and retinoids in combination with compatible oils and antioxidants to provide for improved color stability of the composition.

### BACKGROUND OF THE INVENTION

Standard definitions of ideal skin presently include youthful and resilient skin having uniform color distribution and smooth texture throughout. However, skin is subject to deterioration through, for example, chronoaging. Aging individuals increasingly develop facial fine lines, wrinkles, yellowing or sallowness, sagging, hyperpigmentation, age spots and the general signs of aging.

Skin care compositions containing retinoids, such as retinol, have become quite prominent in recent years. Retinol, also known as Vitamin A, and many of its ester derivatives and retinoid family members including, for example, retinyl propionate, can be effective in reducing fine lines and wrinkles, smoothing skin, and improving uneven skin tone among other benefits when applied topically. Notwithstanding the benefits of retinoids, the inclusion of retinoid compounds in cosmetic compositions often results in compositions having difficulties with stability.

In addition, many people often look to cosmetic products with skin-lightening benefits for desirable even skin tone. There exists an ever-present need in the industry for new cosmetic skin-lightening agents that have enhanced efficaciousness and stability.

Examples of skin-lightening agents include niacinamide, hydroquinone, and kojic acid. Resorcinols, such as hexylresorcinol, provide further examples of skin-lightening agents that may be used in topical applications for the skin.

Skin care compositions containing skin-lightening agents such as hexylresorcinol have become quite prominent in recent years. However, obtaining cosmetic compositions comprising resorcinols that are color stable is challenging. Color instability is of concern because consumers are sensitive to changes in color appearance of cosmetic compositions that they use, especially in cases where people may believe that color change cues degradation of chemical activeness. The color instability of resorcinols is positively correlated in a nonlinear manner with chemical instability of resorcinols, but not in a proportional fashion. Color stability in compositions with resorcinols tends to deteriorate in a shorter time period than does chemical activity. Trace amounts of chemical degradation in resorcinols due to extrinsic factors including impurities in the case formulation can result in large degradations of color. The issue of color instability in cosmetic compositions is exacerbated when resorcinols and retinoid compounds are both found in the compositions, as it results in greater degradation of color when compared to compositions with each active independently. Thus, the color instability of cosmetic compositions with both resorcinols and retinoids poses even further a difficulty to be overcome.

Therefore, the present inventor has recognized a need to develop a cosmetic composition with improved color stability that includes both oil-soluble alkylresorcinols, and retinoids and/or the latter's ester derivatives. Thus, this invention is directed to a cosmetic composition having an oil-soluble alkylresorcinol in combination with (a) a retinoid, (b) an ester-derivative of a retinoid or (c) a mixture thereof in addition to butylated hydroxytoluene, dilauryl thiodipropionate, and a compatible oil.

### ADDITIONAL INFORMATION

Efforts have been disclosed for incorporating antioxidants into cosmetic compositions with resorcinols and retinoids. U.S. Patent Application Publications US 2017281507, US 2017281508 and US 2017304178 disclose methods of diminishing aging symptoms and/or improving the health of human integuments, and compositions useful thereof comprising a peptide with respective amino acid sequences. These compositions can optionally include antioxidants, retinol, and/or hexyl resorcinol in the claimed inventions.

PCT application WO 2011/047420 reports a topical antimicrobial composition that can include a polymer of 2-propenal, a C₃₋₁₀ alkanediol and optionally comprise resorcinol or retinol.

U.S. Patent Application Publication US 20160256368 reports methods of treating skin to diminish signs of aging and/or improve skin health by topically applying at least two separate compositions optionally comprising antioxidants, retinols, and resorcinols in a sequential, rotating, or alternating fashion.

None of the additional information above describes the combination of antioxidants, including butylated hydroxytoluene and dilauryl thiodipropionate, with resorcinol, retinoid, and a compatible oil to achieve a color stable cosmetic composition to be used for anti-aging and skin-lightening benefits as disclosed in the presently claimed invention.

### SUMMARY OF THE INVENTION

The present inventors have found that certain ratios of antioxidants comprising BHT and dilauryl thiodipropionate in addition to use of a compatible oil carrier enhance the color stability of compositions comprising both retinoid and oil-soluble alkylresorcinol.

Accordingly, the invention relates to an active antioxidant mixture of butylated hydroxytoluene and dilauryl thiodipropionate.

In a first aspect, the present invention is directed to an antioxidant mixture of butylated hydroxytoluene (BHT) and dilauryl thiodipropionate, further comprising oil-soluble alkylresorcinol, retinoid, or a mixture thereof with a compatible oil.

In a second aspect, the present invention is directed to a cosmetic composition which includes: (i) one or more of an oil-soluble, 4-substituted alkylresorcinol compound; (ii) one or more of a retinoid, retinoid ester-derivative, or a blend thereof; (iii) an active antioxidant mixture according to the first aspect of this invention; (iv) a compatible oil; and (v) a cosmetically acceptable vehicle.

All other aspects of the present invention will become more readily apparent upon considering the detailed description and examples which follow.

For the avoidance of doubt, the term “comprising” is meant not to be limiting to any stated elements but rather to encompass non-specified elements of major or minor functional importance. Therefore, the listed steps, elements or options need not be exhaustive. Whenever the words “including” or “having” are used, these terms are meant to be equivalent to “comprising” as defined above.

The term “skin” as used herein includes the skin on the face, neck, chest, back, arms, axilla, buttocks, hands, legs and scalp. Active, as used herein, is meant to include a component that improves a facial or body characteristic after topical application like a skin characteristic and/or benefits the same wherein the active can be, and preferably is, an active in a cream, pump or aerosol spray, serum, lotion, balm, deodorant, gel or wash composition. In an especially preferred embodiment, the composition of this invention is a leave-on composition and the actives are retinoid compounds and oil-soluble alkylresorcinols. Remains color stable, as used herein, describes a cosmetic composition that possesses a ΔE of ten (10) or less based on L*, a* and b* color differences taken on a Hunter Lab spectrophotometer. Generally, ΔE≤10 is acceptable, ΔE between 10 to 20 is negotiable and ΔE>20 is unacceptable. The term “compatible” as used herein refers to an oil possessing a Hansen total solubility parameter value within the range of 16 to 22. Hansen total solubility parameters is determined based on three Hansen parameters, namely δₜ (defines energy from dispersion forces between molecules), δₚ (defines energy from dipolar intermolecular forces between molecules), and δₙ (defines energy from hydrogen bonds between molecules). δₜ values are obtained according to the CRC Handbook of Solubility Parameters and other Cohesion Parameters, Second Edition by Allan F. M. Barton.

Except in the examples, or where otherwise explicitly indicated, all numbers in this description indicating amounts of material are to be understood as modified by the word “about.” All amounts are by total weight of the composition, unless otherwise specified.

### DETAILED DESCRIPTION OF THE INVENTION

The resorcinol compounds comprised in the cosmetic compositions of the present invention are oil-soluble alkylresorcinols. Illustrated but not limiting examples of oil-soluble, 4-substituted alkylresorcinols include 4-hexyl resorcinol, 4-phenylethyl resorcinol, 4-cyclopentyl resorcinol, 4-cyclohexylresorcinol, and mixtures thereof. The composition comprises 0.001 to 10%, preferably 0.01 to 5%, most preferably 0.01 to 1% by weight of the alkylresorcinol. In an embodiment of the invention, the oil-soluble alkylresorcinol used in the invention is 4-hexyl resorcinol.

The inventive compositions contain a retinoid, which is selected from retinyl esters, retinol, retinal and retinoic acid, preferably retinal or retinyl ester. Examples of retinyl esters suitable for use in the invention include but are not limited to: retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl taurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, and retinyl oleate. The preferred ester for use in the present invention is selected from retinyl palmitate, retinyl acetate, retinyl propionate and mixtures thereof. Retinyl linoleate and retinyl oleate are also preferred. Retinol or retinyl ester is employed in the inventive composition in an amount from 0.001 to 10%, preferably in an amount from 0.01 to 1%, most preferably in an amount from 0.01 to 0.5%.

Now it has been found that use of a compatible oil in compositions with alkylresorcinol, retinoid, and antioxidants can unexpectedly contribute to improved color stability. It has surprising been found that oils with a Hansen total solubility parameter (δₜ) value within the range of 16 to 22, preferably 17 to 19, are most compatible and thus suitable for use in such cosmetic compositions. Illustrative examples of suitable oils for use in the present invention include isopropyl myristate (δₜ=17.5), caprylic/capric triglycerides (δₜ=18.9), and isopropyl palmitate (δₜ=17.2). Compatible oils are employed in the inventive composition in an amount from 13 to 30%, preferably in an amount from 14 to 28%, most preferably in an amount from 15 to 25% based on the total weight of the composition.

It has further been found that when a retinoid and oil-soluble alkylresorcinol is added in combination with identified ratios of butylated hydroxytoluene (BHT), and dilauryl thiodipropionate, which is commercially available under the trademark Tinogard DA from the supplier BASF, to a cosmetically acceptable vehicle to the compositions of this invention, increased color stability is unexpectedly observed when compared to controls of the same vehicle.

The total weight percent of antioxidants comprised in the present invention is preferably from 0.01 to 4 wt %, more preferably, from 0.02 to 3 wt %, and most preferably, from 0.05 to 2 wt %. The minimum total weight percent of antioxidants comprised in the present invention is preferably 0.15 wt % based on the total weight of the composition.

In one embodiment, a 0.8:4.2 to 4.2:0.8 weight ratio of BHT to dilauryl thiodipropionate in addition to a cosmetic composition with resorcinols and retinoids is preferred.

In another embodiment, a 0.8:2.2 to 2.2:0.8 weight ratio of BHT to dilauryl thiodipropionate is more preferred.

In still another embodiment, a 0.8:1.2 to 1.2:0.8 weight ratio of BHT to dilauryl thiodipropionate is most preferred.

Color stability is determined through the use of LabScan XE equipment (Hunter Associates Laboratory, Inc. Reston, Va.). LabScan XE measures color data. Samples at different storage stages housed in vials or in measurement cells are loaded onto the LabScan XE measurement port. By following the measurement procedure specified in the associated instrument menu and computer software, colors (a*, b* and L*) are measured and calculated to result in a color change data (ΔE) value. For reference, a ΔE of approximately three is the threshold where one could visibly detect a color change from the original sample.

Cosmetic compositions of the present invention may also comprise cosmetically acceptable vehicle in addition to a compatible oil to act as a diluent, dispersant or carrier for the skin benefit agents in the composition, so as to facilitate its distribution when the composition is applied to the skin. This cosmetically acceptable vehicle may be aqueous, anhydrous or an emulsion. Oily carriers in the presence of water and an emulsifier will form emulsion systems as carriers. Preferably, the compositions are aqueous or an emulsion, especially water-in-oil or oil-in-water emulsion, preferentially oil-in-water emulsion. The cosmetic compositions ordinarily will be in but are not limited to cream or lotion form.

In the most preferred embodiment, water is the carrier. Amounts of water may preferably range from 5 to 85%, more preferably, from 15 to 75%, and most preferably, from 30 to 70%, based on the total weight of the total cosmetic composition and including all ranges subsumed therein.

Besides water, suitable carrier classes include silicones, polyhydric alcohols, fatty acids, hydrocarbons, triglycerides, waxes and thickening agents.

Silicones may be categorized into the volatile and nonvolatile variety. Amounts may range, for example, from 0.01 to 25%, more preferably from 0.1 to 20% by weight of the composition. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 4 to 5, silicon atoms.

Nonvolatile silicones useful in this composition include polyalkyl siloxanes, polyalkylaryl siloxanes and polyether siloxane copolymers. The essentially nonvolatile polyalkyl siloxanes useful herein include, for example, polydimethyl siloxanes with viscosities from about 5×10⁻⁶ to 0.1 m²/s at 25° C. Emulsifying and non-emulsifying silicone elastomers are also suitable for use in the cosmetic composition of this invention.

Conventional humectants, generally of the polyhydric alcohol-type materials, may be used in the present invention. Typical polyhydric alcohols include glycerol (i.e., glycerine or glycerin), propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The most preferred embodiment includes glycerin, propylene glycol or a mixture thereof. The amount of humectant employed may range anywhere from 0.5 to 25%, preferably between 1 and 20% by weight of the cosmetic composition.

Fatty acids may also be useful carriers. The term “fatty” refers to carbon chain lengths ranging from 10 to 30 carbon atoms. Illustrative of this category are pelargonic, lauric, myristic, palmitic, stearic, isostearic, hydroxystearic, oleic, linoleic, ricinoleic, arachidic, behenic, erucic acids and combinations thereof. When used, from 0.01 to 5% by weight fatty acid is present in the composition.

Waxes and wax esters such as beeswax, spermaceti wax, tribehenin wax, carnauba wax and candelilla wax are also suitable for use in the present invention as carriers. The amount of wax and wax esters employed may range from 0.01 to 10%, preferably from 0.1 to 8% by weight of the composition.

Triglycerides are another group of materials useful as carriers. Illustrative but not limiting examples are sunflower seed oil, cotton oil, canola oil, grapeseed oil, soybean oil, castor oil, borage oil, olive oil, shea butter, jojoba oil and mixtures thereof. Mono- and di-glycerides may also be useful. Illustrative of these categories are glyceryl monostearate and glyceryl distearate.

Amounts of triglycerides may range from 0.01 to 10%, preferably from 0.1 to 8% by weight of the composition.

The carriers may comprise one or more thickening agents, preferably from 0.05 to 10%, more preferably from 0.1 to 5%, and even more preferably from 0.25 to 4%, by weight for the composition. Useful thickeners include polysaccharides, which comprise of starches, natural/synthetic gums and cellulosics. Suitable starches include tapioca starch, cornstarch, potato starch, aluminum starch octenylsuccinate and sodium hydroxypropyl starch phosphate. Suitable gums include xanthan, sclerotium, pectin, karaya, arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers functioning as thickening agents are also suitable for use, including polyacrylamides, Carbomers, taurate copolymers, and acrylate-based polymers.

Emulsifiers are preferably present in the cosmetic composition of the present invention. Emulsifiers suitable for use in the present invention will have an HLB from 2.5 to 17. The HLB of the emulsifier used when oil-continuous emulsions are desired will be 2.5 to 7.5, and preferably, from 3 to 6.5, and most preferably, from 3 to 6, including all ranges subsumed therein. The HLB of the emulsifier used when water-continuous emulsions are desired will be 8 to 17, and preferably, from 8.5 to 15, and most preferably from 9 to 14, including all ranges subsumed therein.

Total concentration of the emulsifier may range from 0.1 to 20%, and preferably, from 1 to 10%, and most preferably, from 1 to 8% by weight of the composition, including all ranges subsumed therein. The emulsifier may be selected from the group consisting of anionic, nonionic, cationic and amphoteric actives. Particularly preferred nonionic actives are those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di- C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Cationic emulsifiers that may be used include, for example, palmitamidopropyltrimonium chloride, distearyldimonium chloride and mixtures thereof. Useful amphoteric emulsifiers include cocoamidopropyl betaine, C₁₂-C₂₀ trialkyl betaines, sodium lauroamphoacetate, and sodium laurodiamphoacetate or a mixture thereof.

Other generally preferred emulsifiers include glyceryl stearate, glycol stearate, stearamide AMP, PEG-100 stearate, as well as emulsifying/thickening additives like hydroxyethylacrylate/sodium acryloyldimethyl taurates copolymer/squalane and mixtures thereof.

Fragrances, actives, fixatives and abrasives may optionally be included in cosmetic compositions of the present invention. Actives suitable for use in this invention are meant to include but not be limited to opacifiers, colorants, humectants, emollients, occlusive agents, plant extracts, optical agents, skin lightening agents, anti-inflammatory agents, anti-acne agents, sunscreens, photostabilizers, surfactants, wrinkle reducing agents, desquamation promoters, exfoliating agents, mixtures thereof or the like. Each of these substances may range from 0.05 to 5%, preferably between 0.1 and 3% by weight.

Hydrocarbons may be optionally included in the cosmetic of the present invention. Suitable hydrocarbons may include mineral oil, petrolatum and polyalpha-olefins. Examples of preferred volatile hydrocarbons include polydecanes such as isodecane and isododecane and the C₇-C₈ through C₁₂-C₁₅ isoparaffins.

Cosmetic compositions of the present invention may include vitamins as the desired active. Illustrative vitamins are Vitamin B₂, Vitamin B₃ (niacinamide), Vitamin B₅ (panthenol), Vitamin B₆, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. Total amount of vitamins when present in cosmetic compositions according to the present invention may range from 0.001 to 10%, preferably from 0.01 to 5%, optimally from 0.05 to 2.5% by weight of the cosmetic composition.

Optional actives suitable for use in this invention include resveratrol, saccharide isomerate, ceramides (e.g. Ceramide 1, Ceramide 3, Ceramide 3B and Ceramide 6) and pseudoceramides, allantoin, pentaerythrityl tetra-di-t-butyl hydroxyhydrocinnamate, salicylic acid, pyroglutamic acid (PCA) salt derivatives including zinc PCA and sodium PCA, 12-hydroxystearic acid, petroselinic acid, conjugated linoleic acid, octadecanoic acid, hyaluronic acid and its salt derivatives and mixtures thereof or the like. Such actives, when used, collectively make up from 0.001 to 12% by weight of the cosmetic composition.

A wide selection of botanical extracts may optionally be included in cosmetic compositions of this invention. The extracts may either be soluble in water or oil, carried in a solvent that is hydrophilic or hydrophobic, respectively. In the preferred embodiment, water or ethanol are the extract solvents. Illustrative examples include those extracted from green tea, yarrow, chamomile, licorice, aloe vera, citrus unshui, willow bark, alfalfa, algae, witch hazel, sage, thyme and rosemary, as well as oils such as those derived from sea buckthorn, moringa, argan, avocado, calendula, algal, marula and hemp seed. Soy extracts may be used and especially when it is desirable to include retinol.

Traditional buffers or pH modifiers are also suitable for inclusion to the cosmetic compositions of this invention. These include common additives such as sodium hydroxide, potassium hydroxide, hydrochloric acid, citric acid, triethanolamine and aminomethyl propanol. In a preferred embodiment, the pH of the cosmetic composition of the present invention is from 4 to 8, and more preferably, from 4.25 to 7.75, and most preferably, from 5 to 7.5. Viscosity of the cosmetic composition of this invention is preferably from about 1,000 to about 120,000 cps, and most preferably, from about 5,000 to 80,000 cps, taken under conditions of ambient temperature and a shear rate of 1s⁻¹ with a strain controlled parallel plate rheometer made commercially available from suppliers like T.A. Instruments under the Discovery name. Alternatively, viscosity can also be measured using a Brookfield Viscometer (speed at 20 rpm, spindle 5, heliopath off, for one (1) minute at ambient temperature). The cosmetic composition of the invention can be formulated as a lotion having a viscosity from 4,000 to 10,000 mPas, a fluid cream having a viscosity from 10,000 to 20,000 mPas or a cream having a viscosity from 20,000 to 100,000 mPas or above.

Preservatives can be incorporated into the compositions of this invention as desired to protect against the growth of potentially harmful microorganisms. Cosmetic chemists are familiar with appropriate preservatives and routinely choose them to satisfy preservative tests and product stability tests. Preservative systems should be selected having regard for the use of the composition and possible incompatibilities between the preservatives and other ingredients in the formulation. Exemplary examples of preservatives for compositions of this invention include, without limitations, iodopropynyl butyl carbamate (IPBC), phenoxyethanol, ethylhexylglycerine, 1,2-octanediol, methyl paraben, propyl paraben, imidazolidinyl urea, sodium dehydroacetate, propanediol, alkyl esters of para-hydroxybenzoic acid, hydroxyacetophenone, DM DM hydantoin derivatives, climbazole, propionate salts, and a variety of quaternary ammonium compounds.

Preservatives are preferably employed in amounts ranging from 0.01 to 2% by weight of the composition, including all ranges subsumed therein.

When making the cosmetic composition of the present invention, the desired ingredients are mixed via conventional methods. If applicable, the oil phase and water phases are separately mixed using standard mixers and mixing blades and heated to the same temperature usually from about 25 to about 85° C. under atmospheric pressure. Upon reaching the desired temperature, the oil phase is added to the water phase to create an emulsion. Other ingredients in the cosmetic composition are subsequently added and mixed in no particular order.

The cosmetic composition of this invention is a composition suitable for topical application to human skin, including leave-on and wash-off products. Preferably the term encompasses a fluid liquid, and particularly a moisturizer rather than a make-up product. Most preferred are leave-on compositions. The term “leave-on” as used with reference to compositions herein means a composition that is applied to or rubbed on the skin and left thereon.

Many types of packaging can be used to store and deliver the composition of the present invention. The selection of packaging is dependent upon the personal care end-use and the viscosity of the composition itself. As an example, leave-on lotions and creams for skin typically employ plastic containers with an opening at a dispense end covered by an appropriate closure. Conventional closures include flip-top hinged lids, screw-caps and non-aerosol pumps. As another example, appropriate packaging to be used for antiperspirants, deodorants and depilatories include a container with a roller-ball applicator on a dispensing end if the composition is fluid and of a thinner viscosity. If the composition is in a stick format, a container with a propel-repel mechanism wherein the stick is fixed on a platform towards a dispensing orifice is appropriate. If the composition is in an aerosol format, then metallic cans pressurized by a propellant and having a spray nozzle is appropriate. In general, patches, bottles, tubes, roller-ball applicators, squeeze containers or lidded jars are preferred.

The following examples will more fully facilitate an understanding of the present invention. The examples are not intended to limit the scope of the claims.

### EXAMPLES

All samples were made by mixing the mentioned ingredients under conditions of moderate sheer, ambient temperature to about 85° C., and atmospheric pressure.

### Example 1: Full Formulation with Retinyl Propionate (RP),Hhexylresorcinol (HR), Antioxidants, and Different Oils

| | |
|---|---|
| Ingredient | Wt. % |

Part A: Water Phase (main mixer)

| | |
|---|---|
| DI Water | Balance |
| Glycerin | 7.0 |
| Pentylene Glycol | 3.0 |
| Thickener | 0.4 |
| Disodium EDTA | 0.1 |
| Tween 20 | 0.5 |

Part B: Oil Phase

| | |
|---|---|
| Hexylresorcinol | 0.4 |
| Emulsifier | 2.5 |
| Vitamin E Acetate | 0.2 |
| Oil | 15.0 |
| BHT | 0.1 |
| Dilauryl thiodipropionate (Tinogard DA) | 0.1 |
| Dimethicone | 6.3 |
| 12-hydroxystearic acid | 0.01 |
| Thickener | 0.8 |

Part C: Active

| | |
|---|---|
| Retinyl Propionate | 0.3 |

### Example 2: Full Formulation as in Example 1 with Varying Amounts of Oil

| | ΔE | | |
|---|---|---|---|
| | Week 4 | Week 8 | Week 12 |
| 10% oil | 10.72 | 19.21 | 31.3 |
| 15% oil | 8.0 | 11.5 | 13.5 |

As can be seen from the data, oil used consistent with the invention unexpectedly stabilizes a formulation with both HR and RP. The formulation with more than 13% oil yields lower ΔE values over time than that with 10% oil.

### Example 3: Full Formulation as in Example 1 with Different Oils

| δₜ of oil in | ΔE | | |
|---|---|---|---|
| formulation | Week 4 | Week 8 | Week 12 |
| (a) 14.4 | 14.9 | 22.9 | 26.8 |
| (b) 17.5 | 6.1 | 9.9 | 14.2 |
| (c) 18.9 | 8 | 11.5 | 13.5 |
| (c) 24.7 | 40 | 43.1 | 46.7 |

Sample (a) is C₁₂₋₁₃ alkane oil with a δₜ of 14.4. Sample (b) is Isopropyl Myristate with a δₜ of 17.5. Sample (c) is Caprylic/Capric Triglyceride having a δₜ of 18.9. Sample (d) is Benzyl Alcohol having a δₜ of 24.7. All δₜ data are collected from the CRC Handbook of Solubility Parameters and Other Cohesion Parameters, Second Edition (by Allan F. M Barton). From the data in this example, it is demonstrated that oils with δₜ values consistent with the invention yield lower ΔE values (i.e. superior color stability) over time.

### Example 4: Base O/W Formulation

| | |
|---|---|
| Ingredient | Wt. % |

Part A: Water Phase (main mixer)

| | |
|---|---|
| DI Water | Balance |
| Glycerin | 3.0 |
| Butylene Glycol | 2.0 |
| Thickener | 0.8 |
| Disodium EDTA | 0.1 |
| Preservative | 0.2 |

Part B: Oil Phase

| | |
|---|---|
| Hydrocarbon oil | 0-20 |
| Ether oil | 0-20 |
| Stearic acid | 0.1 |
| Emulsifier | 1.5 |
| Climbazole | 0.3 |

### Example 5: Base IO/W Formulation as in Example 4 with Retinyl Propionate (RP), Hexylresorcinol (HR), BHT, and Tinogard DA

| | |
|---|---|
| Ingredient | Wt. % |

Part A: Water Phase (main mixer)

| | |
|---|---|
| DI Water | Balance |
| Glycerin | 3.0 |
| Butylene Glycol | 2.0 |
| Thickener | 0.8 |
| Disodium EDTA | 0.1 |
| Preservative | 0.2 |

Part B: Oil Phase

| | |
|---|---|
| Hexylresorcinol | 0.40 |
| Hydrocarbon oil | 0-20 |
| Ether oil | 0-20 |
| Stearic acid | 0.1 |
| Emulsifier | 1.5 |
| Climbazole | 0.3 |
| Dilauryl thiodipropionate (Tinogard DA) | 0.1 |
| BHT | 0.1 |

Part C: Active

| | |
|---|---|
| Retinyl Propionate | 0.3 |

### Example 6: Color Stability Data with O/W Formulation from Example 4 with Retinyl Propionate, Hexylresorcinol, and Iterations of Individual aAtioxidants, then 1:1 BHT:Tinoaard DA Mixture

| | ΔE | | | |
|---|---|---|---|---|
| | Week 2 | Week 4 | Week 8 | Week 12 |
| O/W with 0.01% BHT | 6.1 | 7.7 | 11.8 | 14.1 |
| O/W with 0.05% BHT | 5.0 | 6.8 | 10.7 | 13.4 |
| O/W with 0.1% BHT | — | 5.4 | 8.3 | 10.2 |
| O/W with 0.2% BHT | 4.3 | 5.2 | 8.6 | 11.4 |
| O/W with 0.01% Tinogard DA | 5.4 | 7.8 | 11.4 | 14.1 |
| O/W with 0.05% Tinogard DA | 5.7 | 7.4 | 11.1 | 13.8 |
| O/W with 0.1% Tinogard DA | — | 5.0 | 8.2 | 11.7 |
| O/W with 0.2% Tinogard DA | 6.9 | 6.8 | 9.4 | 11.6 |
| O/W with 0.05% BHT + | 5.6 | 5.6 | 8.7 | 11.4 |
| 0.05% Tinogard DA | | | | |
| O/W with 0.1% BHT + | 4.5 | 4.2 | 6.6 | 8.9 |
| 0.1% Tinogard DA | | | | |
| O/W with 0.2% BHT + | 6.3 | 5.0 | 7.1 | 9.1 |
| 0.2% Tinogard DA | | | | |

Compared with HR and RP samples with only individual antioxidants, it is surprisingly shown that compositions with BHT and Tinogard DA exhibit lower ΔE values over time notwithstanding that all sample compositions begin at 2 weeks with approximately similar ΔE values.

### Example 7: Color Stability Data with O/W Formulation from Example 4 with Hexylresorcinol, Retinyl Propionate with 4:1 and 1:4 Antioxidant Mixture Ratios

| | ΔE | | | |
|---|---|---|---|---|
| | Week 2 | Week 4 | Week 8 | Week 12 |
| O/W with 4:1 BHT:Tinogard | 3.2 | 4.6 | 8.2 | 10.9 |
| DA (total 0.2%) | | | | |
| O/W with 1:4 BHT:Tinogard | 3.7 | 4.4 | 8.9 | 11.5 |
| DA (total 0.2%) | | | | |
| O/W with 0.1% BHT + | 4.5 | 4.2 | 6.6 | 8.9 |
| 0.1% Tinogard DA | | | | |
| O/W with 0.16% BHT + | 3.2 | 4.6 | 8.2 | 10.9 |
| 0.04% Tinogard DA | | | | |
| O/W with 0.04% BHT + | 3.7 | 4.4 | 8.9 | 11.5 |
| 0.16% Tinogard DA | | | | |

The data unexpectedly demonstrates that 4:1 and 1:4 ratios of BHT to Tinogard DA in the O/W base formulation with HR and RP exhibit lower ΔE values over time as well, and therefore, superior color stability.

### Example 8: Color Stability Assessment with O/W Formulation from Example 4 with Hexylresorcinol, Retinyl Propionate and Other Antioxidants

| | ΔE | | | |
|---|---|---|---|---|
| | Week 2 | Week 4 | Week 8 | Week 12 |
| O/W with 0.1% Tinogard DA + | 6.7 | 7.0 | 10.1 | 12.7 |
| 0.01% Tinogard TT | | | | |
| O/W with 0.1% Tinogard DA + | 6.5 | 6.9 | 10.0 | 12.6 |
| 0.05% Tinogard TT | | | | |
| O/W with 0.1% Tinogard DA + | 6.8 | 7.0 | 10.1 | 12.8 |
| 0.2% Tinogard TT | | | | |
| O/W with 0.1% Tinogard DA + | — | 5.0 | 8.2 | 11.7 |
| 0% NaHSO₃ | | | | |
| O/W with 0.1% Tinogard DA + | — | 5.7 | 8.4 | 11.6 |
| 0.001% NaHSO₃ | | | | |
| O/W with 0.1% Tinogard DA + | — | 8.6 | 11.5 | 13.9 |
| 0.01% NaHSO₃ | | | | |
| O/W with 0.1% Tinogard DA + | — | 11.9 | 18.1 | 23.0 |
| 0.1% NaHSO₃ | | | | |
| O/W with 0.1% Tinogard DA + | — | 8.1 | 14.2 | 18.2 |
| 0.1% BHT + 0.1% NaHSO₃ | | | | |
| O/W with 0.1% Salicylic acid | — | 9.0 | 13.7 | 16.1 |
| O/W with 0.1% Rosamox | — | 7.1 | 10.2 | 12.2 |
| O/W with 0.1% Green Tea Leaf | — | 7.0 | 10.5 | 12.7 |

Tinogard TT is the phenolic antioxidant under the chemical name Pentaerythrityl Tetra-di-t-butyl Hydroxyhydrocinnamate that is made commercially available from BASF. Rosamox is a natural antioxidant sold under the chemical name Rosmarinus officinalis (Rosemary) Leaf Extract that is made commercially available from Kemin.

The results demonstrate that numerous other antioxidants were tested in the base formulation presented in Example 1 for purposes of color stability assessment with alkylresorcinols and retinoids. These other antioxidants alone or in combination with additional antioxidant candidates are surprisingly not as effective as that seen in the BHT-Tinogard DA combination consistent with this invention.

1. A cosmetic composition comprising:
(a) an oil-soluble alkylresorcinol;
(b) a retinoid;
(c) an oil where the oil has a Hansen total solubility parameter from 16 to 22; and
(d) an active mixture of BHT and dilauryl thiodipropionate, wherein the weight ratio of BHT to dilauryl thiodipropionate is from 0.8:4.2 and 4.2:0.8.

2. The cosmetic composition according to claim 1, wherein antioxidants are present at a minimum total weight percent of 0.15% by weight of the cosmetic composition.

3. The cosmetic composition according to claim 1, wherein the alkylresorcinol is selected from the group consisting of 4-hexyl resorcinol, 4-phenylethyl resorcinol, 4-cyclopentyl resorcinol, 4-cyclohexylresorcinol and mixtures thereof, and further wherein the composition comprises 0.001 to 10% by weight of alkylresorcinol or a mixture thereof.

4. The cosmetic composition according to claim 1, wherein the retinoid comprises of retinyl esters, retinol, retinal ester, retinoic acid, retinal ester, retinyl ester, and mixtures thereof, and further wherein the composition comprises 0.001 to 10% by weight of the retinoid or mixture thereof.

5. The cosmetic composition according to claim 1, wherein the composition comprises a 0.8:2.2 to 2.2:0.8 ratio of BHT to dilauryl thiodipropionate.

6. The cosmetic composition according to claim 1, wherein the composition comprises a 0.8:1.2 to 1.2:0.8 ratio of BHT to dilauryl thiodipropionate.

7. The cosmetic composition according to claim 1, wherein the composition comprises an oil with a Hansen total solubility parameter from 17 to 19.

8. The cosmetic composition according to claim 1, wherein the composition comprises 14 to 28% by weight oil.

9. The cosmetic composition according to claim 1, wherein the composition comprises 15 to 25% by weight oil.

10. The cosmetic composition according to claim 1, wherein the composition comprises an oil that is selected from isopropyl myristate, isopropyl palmitate, caprylic/capric triglycerides, or mixtures thereof.

11. The cosmetic composition according to claim 1, wherein the composition further comprises at least one of niacinamide, 12-hydroxystearic acid, a sunscreen, wrinkle-reducing agent, or a mixture thereof.

12. An active antioxidant mixture consisting essentially of butylated hydroxytoluene (BHT) and dilauryl thiodipropionate, wherein the weight ratio of BHT to dilauryl thiodipropionate is from 0.8:4.2 to 4.2:0.8, and further wherein a cosmetic composition comprises 0.2 to 4 wt % by total weight of the active mixture.

13. The active antioxidant mixture according to claim 12, wherein the active mixture further comprises an alkylresorcinol or mixture thereof in an amount from 0.1 to 300% by total weight of the active mixture.

14. The active antioxidant mixture according to claim 12, wherein the weight ratio of BHT to dilauryl thiodipropionate is from 0.8:2.2 to 2.2:0.8.

15. The cosmetic composition according to claim 1, wherein the composition comprises 0.01 to 5% by weight of alkylresorcinol.

16. The cosmetic composition according to claim 1, wherein alkylresorcinol is 4-hexyl resorcinol.

17. The cosmetic composition according to claim 1, wherein the composition comprises 0.01 to 1% by weight of retinoid.

18. The cosmetic composition according to claim 1, wherein the retinoid is selected from retinyl palmitate, retinyl acetate, retinyl propionate, and mixtures thereof.
